(19) United States
(12) Patent Application Publication
(10) Pub. No.: US 2017/0239335 A1
(43) Pub. Date: Aug. 24, 2017

(54) STABLE INJECTABLE COMPOSITION OF PHARMACEUTICALLY ACTIVE AGENTS AND PROCESS FOR ITS PREPARATION

(71) Applicant: Piramal Enterprises Limited, Mumbai (IN)

(72) Inventors: Vandana SONAVARIA, Mumbai (IN); Kamal Kumar UPADHYAY, Mumbai (IN)

(21) Appl. No.: 15/519,435

(22) PCT Filed: Oct. 15, 2015
(86) PCT No.: PCT/IB2015/057920

§ 371 Date: Apr. 14, 2017

(30) Foreign Application Priority Data
Oct. 16, 2014 (IN) 3295/MUM/2014

(51) Int. Cl. A61K 38/49; A61K 47/10; A61K 31/675; A61K 47/26; A61K 9/08; A61K 9/00; A61K 38/12

(52) U.S. Cl. CPC A61K 38/49; A61K 9/0019; A61K 47/10; A61K 38/12; A61K 47/26; A61K 9/08; A61K 31/675; C12Y 304/21073

(57) ABSTRACT

The present invention relates to a stable, non-aqueous and ready-to-use injectable composition of a pharmaceutically active agent a pharmaceutically active agent or a pharmaceutically acceptable salt or a co-crystal thereof. The present invention also relates to a process for the preparation of the stable, non-aqueous and ready-to-use injectable composition of pharmaceutically active agent involving use of a non-solvent solvent system suitable for preparing a stabilized injectable composition comprising a pharmaceutically active agent a pharmaceutically active agent or a pharmaceutically acceptable salt or a co-crystal thereof. It is not required to reconstitute the injectable composition of pharmaceutically active agent with water prior to administration, thereby rendering it an easy-to-use injectable composition.

### FIELD OF THE INVENTION

The present invention relates to a stable, non-aqueous and ready-to-use injectable composition of a pharmaceutically active agent or a pharmaceutically acceptable salt or a co-crystal thereof; and processes for its preparation.

### BACKGROUND OF THE INVENTION

The choice of delivery routes for therapeutically active agents is wide. However, for an appropriate delivery route for a given therapeutically active agent, certain key factors are given consideration to, which include, drug properties, clinical application and patient compliance. Thus, in meeting the challenges of viable delivery systems for a therapeutically active agent, one needs to consider not only viability of drug delivery but also the related aspects of attainability and reliability. Parenteral administration is one of the most used delivery route to obtain systemic delivery of pharmaceutically active agents such as small molecules, peptides or proteins. These are frequently formulated in aqueous solutions. However, pharmaceutically active agents such as small molecule drugs, peptides and proteins; particularly, those which are hydrophilic in nature are generally unstable in aqueous environment as they commonly undergo degradation. Solubility and stability plays a major role for parenteral compositions. For addressing the stability problems of pharmaceutically active agents such as peptides, proteins and even certain small molecule drugs in aqueous environment, such active agents are commonly formulated as a solid by lyophilization and reconstituted with a sterile diluent prior to administration. Representative examples of pharmaceutically active agents that are marketed as lyophilized powder for injection include: (i) Urokinase is commercially available as Kinlytic™ (Microbix Biosystems), which is a sterile lyophilized white powder containing 250,000 international units urokinase per vial, mannitol (25 mg/vial), albumin (Human) (250 mg/vial), and sodium chloride (50 mg/vial); (ii) Protein C Concentrate (Human), commercially available as Ceprotin®, which is a natural protein that is made in the liver and is present in the blood. Ceprotin is used to treat patients with severe congenital protein C deficiency for the prevention and treatment of: venous thrombosis (blood clot in the vein), and purpura fulminans (blood spots, bruising and discoloring to skin as a result of clotting of small blood vessels in the skin); (iii) Coagulation Factor IX (Recombinant), commercially available as Alprolix™, is a Fc Fusion Protein. Alprolix™ is a sterile, non-pyrogenic, preservative-free, white to off-white, lyophilized powder to cake for reconstitution with the provided diluent, for intravenous injection and is indicated in adults and children with hemophilia B (congenital Factor IX deficiency) for: control and prevention of bleeding episodes in perioperative management, and in routine prophylaxis to prevent or reduce the frequency of bleeding episodes; (iv) Acthrel® (Corticorelin ovine triflutate for injection) is a sterile, nonpyrogenic, lyophilized white cake powder, containing corticorelin ovine triflutate, a trifluoroacetate salt of a synthetic peptide that is used for the determination of pituitary corticotroph responsiveness; (v) Geref® (Sermorelin acetate) is a sterile, non-pyrogenic, lyophilized powder intended for subcutaneous injection after reconstitution with sodium chloride injection, USP. Geref® (sermorelin acetate for injection) increases plasma growth hormone (GH) concentration by stimulating the pituitary gland to release GH; (vi) Cubicin® (Daptomycin) is supplied as a sterile, lyophilized 500 mg or 350 mg cake that must be reconstituted with sodium chloride prior to use. Daptomycin is a lipopeptide antibiotic which is used in the treatment of complicated skin and skin structure infections (cSSSI) caused by susceptible isolates of the following Gram-positive bacteria; (vii) Tigecycline, commercially available as Tygacil® for injection as lyophilized powder indicated for the treatment of bacterial infections; (viii) Bortezomib, commercial available as Velcade®, is available for intravenous injection (IV) use only and each single dose vial contains 3.5 mg of bortezomib as a sterile lyophilized powder indicated for the treatment of multiple myeloma patients who have received at least 1 prior therapy; (ix) Caspofungin acetate, commercially available as Cancidas® for injection as lyophilized powder indicated for the treatment of fungal infections; and (x) Fosaprepitant dimeglumine, commercially available as Emend® for intravenous injection as lyophilized powder, indicated for the treatment of nausea and vomiting that may be caused by surgery or cancer chemotherapy.

Compositions including lyophilized compositions for pharmaceutically active agents are known in the art. WO2014041425 discloses a lyophilized daptomycin composition comprising an additive selected from the group consisting of pharmaceutically acceptable antioxidants, pharmaceutically acceptable organic acids and pharmaceutically acceptable salts thereof, pharmaceutically acceptable glucose derivatives and pharmaceutically acceptable salts thereof, and combinations thereof.

WO2011063419 discloses a solid daptomycin preparation with improved reconstitution time and stability profile.

WO2014045296 discloses a lyophilized pharmaceutical composition comprising antibacterial agent, daptomycin and tocopheryl phosphate hydrolysate mixture with improved reconstitution time for parenteral administration and also discloses a process of preparation thereof.

U.S. Pat. No. 4,244,943 discloses a method for preparing a stable urokinase injection by lyophilization of urokinase which comprises lyophilizing an aqueous solution containing urokinase, human serum albumin and one or more amino acid compounds selected from polar amino acids and salt thereof.

WO 97/04801 disclosed lyophilized compositions that can be reconstituted to generate high protein-concentration liquid compositions without apparent loss of stability. However, the potential issues associated with the high viscosity of the reconstituted compositions are not addressed.

U.S. Pat. No. 5,952,300 discloses a pharmaceutical composition comprising caspofungin as an active ingredient, a pharmaceutically acceptable amount of an excipient such as a sucrose/mannitol mixture to form a lyophilized cake and a pharmaceutically acceptable amount of an acetate buffer effective to provide a pH of between about 4 and 7.

EP2170362 discloses a lyophilized anti-fungal composition comprising; (a) caspofungin, or a pharmaceutically acceptable salt thereof, in an effective amount; (b) one or more non-reducing sugars having a glass transition temperature T_{g}(s) of at least about 90° C.; and (c) an acetate buffer in an amount effective to provide a pH in a range of from about 5 to about 7; wherein the weight ratio of one or more non-reducing sugars to caspofungin is in a range of about 1.1:1 to about 10:1; the composition has a moisture content of about 0.8 weight % or less; and the composition has a glass transition temperature T_{g}(c) of at least about 55° C. The composition needs reconstitution with water prior to use in preventing or treating fungal infections.

EP2049142 discloses pharmaceutical composition comprising the compound, caspofungin as an active ingredient, specific bulking agents and without an additional pH modifier, which compositions are liquid or solid, e.g. lyophilized compositions.

EP2644189 discloses bortezomib composition with improved stability, and particularly storage-stable multi-dose liquid bortezomib compositions. The examples disclosed in this patent document illustrate that water is an essential ingredient of the composition.

It is evident from the above discussion that stability problems of pharmaceutically active agents such as peptides, proteins and even certain small molecule drugs; in aqueous environment is considerably addressed by providing the active agents as lyophilized compositions, which have very low moisture content and require to be reconstituted with an aqueous medium prior to their administration. However, the presence of water or any other aqueous medium in the composition can lead to deterioration of the pharmaceutically active agent because of hydrolysis. Therefore, it is desirable to develop a stable composition for the pharmaceutically active agents that are hydrophilic in nature and for the purpose non-aqueous based compositions would be appropriate. To overcome stability problem, it is essential to find a non-aqueous solvent system in which the pharmaceutically active agents have adequate solubility and stability.

Thus, there exists a need for the development of a new or an improved composition for pharmaceutically active agents such as peptides, proteins and certain small molecule drugs that would prevent degradation, yet increase solubility and stability of the active agents. Moreover, there is a need to provide a stable and ready-to-use injectable composition of the pharmaceutically active agents to improve patient compliance.

As stated above use of aqueous solution in case of compositions of certain small molecules, proteins and peptide based actives has been a challenging task due to degradation and impurity generation, which in turn makes the injection preparation unstable.

In consideration of the need as indicated above, inventors of the present invention have done extensive research and conducted several experiments to develop a stable, non-aqueous and ready-to-use injectable composition of pharmaceutically active agents, without a need to reconstitute with water prior to administration, thereby rendering the composition according to the present invention an easy-to-use injectable composition. The inventors have also provided a simple and cost-effective process for preparation of the stable, non-aqueous and ready-to-use composition of the pharmaceutically active agents.

### SUMMARY OF THE INVENTION

In one aspect, the present invention relates to a stable, non-aqueous and ready-to-use injectable composition of a pharmaceutically active agent or a pharmaceutically acceptable salt or a co-crystal thereof; wherein the said injectable composition comprises:
(i) a pharmaceutically active agent or a pharmaceutically acceptable salt or a co-crystal thereof;
(ii) a non-aqueous solvent system;
(iii) optionally a polyol;
(iv) optionally a pH adjusting agent; and optionally an antioxidant.

In one aspect, the present invention provides stable, non-aqueous and ready-to-use injectable composition of a pharmaceutically active agent or a pharmaceutically acceptable salt or a co-crystal thereof; wherein the said injectable composition comprises:
(i) a pharmaceutically active agent or a pharmaceutically acceptable salt or a co-crystal thereof;
(ii) a non-aqueous solvent system consisting of a primary non-aqueous solvent and optionally one or more secondary non-aqueous co-solvent (s);
(iii) optionally a polyol;
(iv) optionally a pH adjusting agent; and
(v) optionally an antioxidant.

In another aspect, the present invention provides a process for the preparation of a stable, non-aqueous and ready-to-use injectable composition of a pharmaceutically active agent or a pharmaceutically acceptable salt or a co-crystal thereof.

In a further aspect, the present invention provides a method for treating or preventing one or more diseases, disorders or conditions, comprising administering to a subject in need thereof; the composition of the present invention in an amount effective to treat or prevent the conditions, diseases or disorders.

In another aspect, the present invention provides use of a stable, non-aqueous and ready-to-use injectable composition of a pharmaceutically active agent or a pharmaceutically acceptable salt or a co-crystal thereof for the manufacture of a medicament for use in the treatment or prevention of one or more diseases, conditions or disorders.

In another aspect, the present invention provides a stable, non-aqueous and ready-to-use injectable composition of a pharmaceutically active agent or a pharmaceutically acceptable salt or a co-crystal thereof; for use in the treatment of a subject having one or more diseases, conditions or disorders.

In still further aspect, the present invention relates to a pharmaceutical kit comprising: (a) an injectable composition comprising a pharmaceutically active agent or a pharmaceutically acceptable salt or a co-crystal thereof; a non-aqueous solvent system consisting of a primary non-aqueous solvent, optionally one or more secondary non-aqueous co-solvent(s); optionallya polyol; optionally a pH adjusting agent; and optionally an antioxidant; and (b) optionally a package insert comprising instructions for using the said injectable composition.

These and other aspects and advantages of the present invention will be apparent to those skilled in the art from the following description.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

### Definitions

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a” “an” and “the” include plural referents unless the content clearly dictates otherwise.

It should be noted that the term “or” is generally employed in its sense including “and/or” unless the content clearly dictates otherwise.

As used herein, the term “about” means approximately and in the context of numerical values the term “about” can be construed to estimate a value that is ±10% of the value or range recited.

Within the context of the present invention the term “stable” as used herein in reference to the injectable composition of pharmaceutically active agents means that the said composition does not exhibit appreciable degradation upon storage over a set time limit, at a set temperature, and at an identified pH or within the context of the present invention the term “stable” as used herein in reference to the injectable composition of the pharmaceutically active agent or a pharmaceutically acceptable salt or a co-crystal thereof; means that the said composition exhibit a chromatographic purity, where in the impurities identified are within the acceptable limit.

Within the context of the present invention, the term “sterile composition” means one in which essentially all forms of microbial life have been destroyed by an appreciable amount to meet the sterilization criteria outlined in the US Pharmacopeia.

Within the context of the present invention, the term “ready-to-use” or “RTU” as used herein in reference to the injectable composition of a pharmaceutically active agent is a non-aqueous, injectable composition that is stable and is not reconstituted from a lyophilizate. The term “RTU” also encompasses within its scope, non-aqueous, injectable composition that is stable and has been diluted from a concentrated, liquid solution just prior to use.

Within the context of the present invention the term “non-aqueous composition” as used herein means a composition with not more than 2% water content.

The term “non-aqueous solvent” means a non-polar solvent which contain bonds between atoms of similar electronegativity like carbon and hydrogen by which they lack partial charges and do not contain hydrogen attached to oxygen or nitrogen so that they are unable to form hydrogen bonds with themselves. Examples of solvents are selected from the group but not limited to ethylene glycol, polyethylene glycols (PEGs), propylene glycol (PG), dipropylene glycol, tripropylene glycol, polyvinylpyrrolidone (PVP), methoxy propylene glycol (MPEG), glycerol, glycofurol or a mixture thereof.

The term “non-aqueous RTU composition” means the composition is devoid of any water content in the final finished product or during process for preparation of the same. However, a negligible amount i.e. not more than 2% of water or moisture may be present due to external environmental factors which does not have any impact on the physiochemical property, specifically on the stability of the composition.

As used herein, the term “has not been reconstituted from a lyophilizate” means that a solid has not been dissolved or suspended.

The term “pharmaceutically acceptable excipient(s)” as used herein means a diluent, carrier, or composition auxiliary, which is non-toxic, and inert, which does not have undesirable effects on a subject to whom it is administered and is suitable for delivering a therapeutically active agent (e.g. small molecule drug, peptide or protein drug) to the target site without affecting the therapeutic activity of the said active agent.

The term “pharmaceutically acceptable salt” or “pharmaceutically acceptable salt(s)” means salt(s) of the pharmaceutically active agents such as small molecule drug, peptide drugs or protein drugs, which can be prepared by treating the pharmaceutically active agent(s) with an appropriate acid or a base. Examples of pharmaceutically acceptable base addition salts include, but are not limited to, sodium, potassium, calcium, magnesium, ammonium salts or an organic base salt. Examples of pharmaceutically acceptable organic base addition salts include, but are not limited to, those derived from organic bases such as lysine, arginine, guanidine, and the like. Examples of pharmaceutically acceptable acid addition salts include, but are not limited to, those derived from inorganic acids such as hydrochloric acid, nitric acid, phosphoric acid, sulfuric acid and the like, as well as the salts derived from organic acids such as acetic acid, trifluoroacetic acid, propionic acid, oxalic acid, maleic acid, benzoic acid, succinic acid, fumaric acid, phthalic acid, benzenesulfonic acid, p-toluenesulfonic acid, citric acid, tartaric acid, methanesulfonic acid and the like.

The term “co-crystal” refers to a crystalline structure made up of two or more components in a definite stoichiometric ratio, where each component is defined as either an atom, ion, or molecule. The term co-crystal” encompasses within its scope many types of compounds, including hydrates, solvates and clathrates.

The term “composition” or “injectable composition” refers to a unit dose or a multi dose of an active pharmaceutical ingredient and a pharmaceutically acceptable excipient, which can be prepared by the processes described in one or more embodiments of the present invention. In the context of the present invention, the terms “composition”, “injectable compositions” and “non-aqueous, stable and ready-to-use injectable composition” are used interchangeably. In the case of the injectable composition of the present invention, the active pharmaceutical ingredient is a drug or a pharmaceutically active agent such as a peptide drug, a protein drug or a small molecule drug.

The term “polyol” as used herein, refers to an alcohol containing multilple hydroxyl groups. Polyols may comprise, but are not limited to, glycerin, sucrose, lactose, glucose, fructose, arabinose, xylose, ribose, mannose, galactose, dextrose, sorbose, sorbitol, mannitol, maltose, cellobiose, xylitol, or a combination thereof.

The term “stirring” encompasses within its scope, sonication or turbulence or agitation by other means. Therefore the term “stirring” can be interchangeably used with the terms “sonication”, “turbulence” or “agitation”.

As used herein, the term “pH” is a measure of hydrogen ion concentration, as commonly used in the art. Customarily, the pH provides a measure on a scale from 0 to 14 of the acidity or alkalinity of a solution. In the context of the present invention, the pH of the injectable composition of pharmaceutically active agents of the present invention is between about 2.0 and about 13.0.

The term “pH adjusting agent” or “pH adjusting agents” as used herein, includes a substance that adjusts the pH of pharmaceutical compositions to intended pH. Customarily, the pH adjusting agents may include pharmaceutically acceptable acids, bases, or buffering agents. For example, the acids may include, but are not limited to, one or more inorganic mineral acids such as citric, fumaric, gluconic, lactic, malic, metatartaric, tartaric, ascorbic and benzene sulphonic acid and the like. In the context of the present invention, the pH adjusting agent may be a base or a buffering agent. The bases may be one or more inorganic bases or organic bases, including, but not limited to, alkaline carbonate, alkaline bicarbonate, alkaline earth metal carbonate, alkaline hydroxide, alkaline earth metal hydroxide or amine. For example, the inorganic or organic base may be an alkaline hydroxide such as lithium hydroxide, potassium hydroxide, cesium hydroxide, sodium hydroxide or the like; an alkaline carbonate such as calcium carbonate, sodium carbonate or the like; or an alkaline bicarbonate such as sodium bicarbonate or the like; the organic base may also be sodium acetate. The buffering agent can be, but is not limited to an alkali metal salt of an amino acid, aluminum hydroxide, aluminum magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartarate, dibasic sodium phosphate, dipotassium hydrogen phosphate, dipotassium phosphate, disodium hydrogen phosphate, disodium succinate, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium taratrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartarate, sodium acetate, sodium bicarbonate, sodium borate, sodium carbonate, sodium citrate, sodium gluconate, sodium hydrogen phosphate, sodium lactate, sodium phthalate, sodium phosphate, sodium polyphosphate, sodium pyrophosphate, sodium sesquicarbonate, sodium succinate, sodium tartarate, sodium tripolyphosphate, tetrapotassium pyrophosphate, tetrasodium pyrophosphate, tripotassium phosphate, trisodium phosphate, or a mixture thereof.

A relative pH has been measured because it is difficult to measure the absolute pH of a non-aqueous solution due to lack of hydrogen ion activity or concentration. Further, the pH of the composition may vary depending upon the type of instrument and dilution media.

In the context of the invention the term “solvent system” refers to a primary solvent and optionally one or more secondary solvent selected from a group of solvents.

Within the context of the present invention, the term “antioxidants” means a substance which is particularly used because certain compounds suitable for use in compositions of the invention are prone to degradation by autoxidation. Antioxidants may comprise, but are not limited to, acetylcysteine, ascorbyl palmitate, butylated hydroxyanisole (“BHA”), butylated hydroxytoluene (“BHT”), monothioglycerol, potassium nitrate, ascorbic acid or sodium ascorbate, sodium formaldehyde sulfoxylate, sodium metabisulfite, sodium bisulfite, vitamin E or a derivative thereof, propyl gallate, edetate (“EDTA”) (e.g., disodium edetate), diethylenetriaminepentaacetic acid (“DTPA”), triglycollamate (“NT”), DL- or D-α-tocopherol, DL- or D-α-tocopheryl acetate or a combination thereof. Antioxidants may also comprise amino acids such as methionine, histidine, cysteine and those carrying a charged side chain, such as arginine, lysine, aspartic acid, and glutamic acid. Any stereoisomer (e.g., L-, D-, or a combination thereof) of any particular amino acid (e.g., methionine, histidine, arginine, lysine, isoleucine, aspartic acid, tryptophan, threonine and combinations thereof) or combinations of these stereoisomers, is also encompassed within the scope of the term “antioxidant” so long as the amino acid is present either in its free base form or its salt form. The antioxidant, if present, may be added to compositions in accordance with the invention in an amount of up to, for example, 0.05% (w/v), preferably from 0.001 to 1%.

Within the context of the present invention and as used herein the term “pharmaceutically active agent” or “pharmaceutically active agents” can be interchangeably used with the term “drugs”, “therapeutically active agents” or “active agents” and refers to biologically active compounds (or pharmaceutically acceptable salts thereof) having different mechanism of actions that are useful for the treatment or prevention of diseases or disorders in humans or other animals or are otherwise useful in enhancing physical or mental well-being of humans or animals. The term “pharmaceutically active agents” can include the biologically active compounds that are hydrophilic, hydrophobic or amphiphilic in nature but are unstable in aqueous environment. Examples of pharmaceutically active agents include, but are not limited to, peptides (peptide drugs), proteins (protein drugs) and small molecule drugs.

Within the context of the present invention and as used herein, the term “peptide drug” or “peptide drug(s)” refers to synthetic or biological compounds (and salts thereof) containing short chains of amino acids bound together by amide (CONH) linkages that have demonstrated or potential use in treating, preventing, or ameliorating one or more diseases, disorders, or conditions in a subject in need thereof. The term “peptide drugs” is used herein interchangeably with the terms “therapeutic peptides” and “peptides”. Typically, the peptide drugs are short chains of amino acid monomers containing up to 50 amino acids bound together by amide (CONH) linkages and have a molecular weight of less than approximately 5000 Daltons. Peptides can be classified by function and also by synthesis. Some common types of peptides classified by function include hormones, neuropeptides, and alkaloids. When classified by synthesis, peptides can be milk, ribosomal, non-ribosomal, and peptonic. Depending on the number of amino acids, peptides are called dipeptides, tripeptides, tetrapeptides, and conjugated peptides which contain amino acid and prosthetic group such as cyclopeptide, glycopeptide, chromopeptide, lipopeptide, nucleopeptide and phosphopeptide. Representative examples of peptide drugs include, but are not limited to, daptomycin, nesiritide, cetrorelix acetate and combination thereof.

Within the context of the present invention and as used herein the term “protein drug” or “protein drug(s)” refers to hormones, enzymes and/or antibodies that are naturally occurring, recombinant or chemically synthesized large biological molecules or macromolecules comprising a plurality of natural or modified amino acids residues bound together by amide (CONH) linkages. The term “protein drug(s)” is used herein interchangeably with the terms “therapeutic protein(s)” and “protein(s)”. The length of proteins may extend from 51 amino acids up to several thousand amino acids. If the proteins on hydrolysis yield only amino acids, they are called as simple proteins and if, the proteins on hydrolysis yield amino acids and additional products, they are called as conjugated proteins. Derivatives of proteins due to action of heat, enzymes, or chemical reagents are called as derived proteins. Protein are also classified according to shape and solubility as fibrous proteins, globular proteins and membrane proteins. In the context of the present invention, proteins can be classified according to biological function such as hormone, enzyme, transport, storage, contractile, structural, protection or antibody. Representative examples of protein drugs include, but are not limited to, urokinase, streptokinase, prolactin and a combination thereof.

Antibody drug conjugates (ADC) are encompassed within the scope of the present invention. ADC are type of biconjugates. A bioconjugate is a compound in which two molecules are attached with a stable chemical link, at least one of which is a biomolecule; for example, a conjugate of a xenobiotic with some groups such as glutathione, sulfate or glucuronic acid, to make it soluble in water or compartmentalized within the cell. ADCs are complex molecules composed of an antibody (a whole monoclonal antibody or an antibody fragment such as a single-chain variable fragment) linked, via a stable, chemical, linker with labile bonds, to a biological active cytotoxic (anticancer) payload or drug.

Within the context of the present invention and as used herein the term “small molecule drug” or “small molecule drug(s)” refers to therapeutically active compounds (and/or salts thereof) that can bring about a desired and/or beneficial therapeutic effect on a subject in need thereof. Typically, the term “small molecule drug(s)” refers to therapeutically active compound(s) having molecular weight of less than about 3000 Daltons. The small molecule drug can be a therapeutically active compound having molecular weight ranging from about 100 Daltons to about 1500 Daltons or from about 150 Daltons to about 1250 Daltons or from about 300 Daltons to about 1100 Daltons or from about 400 Daltons to about 1000 Daltons. In the context of the present invention, a therapeutic agent, for example, a peptide such as bortezomib having molecular weight of less than 1500 Daltons shall be regarded as a small molecule drug. The small molecule drugs can be selected from the group of agents consisting of anti-cancer agents, anti-bacterial agents, immunomodulating agents, anti-obesity drugs, antidiabetic drugs, anti-fungal agents, anti-viral agents, contraceptives, analgesics, anti-inflammatory agents (e.g. steroids or non-steroidal anti-inflammatory drugs (NSAIDs)), antiemetic drugs, vasodilating agents, vasoconstricting agents, and cardiovascular agents. Particularly, the small molecule drug can include, but not limited to, an anti-cancer agent such as azacitidine, bendamustine, bortezomib, carmustine, cisplatin, carboplatin, cyclophosphide, carmustine, daunorubicine, doxorubicin, etoposide, fludarabine, gemcitabine, melphalan, mitomycin, oxaliplatin, pemetrexed, pentostatin, streptozocin, thiotepa, topotecan or vinblastine; a cytoprotective agent such as amifostine; an anti-bacterial agent such as tigecycline, doxycycline, chloramphenicol, azhithromycin or cefazolin; an anti-fungal agent such as caspofungin, micafungin, anidulafungin or voriconazole; an anti-viral agent such as acyclovir or ganciclovir; an anti-psychotic drug such as thiothixene or midazolam; an anti-ulcer agent such as esomeprazole, lansoprazole or pantoprazole; analgesic such as metamizole, hydromorphone or remifentanil; anti-inflammatory agent such as hydrocortisone, methylprednisolone, indomethacin, ketoprofen or parecoxib; an immunomodulating agent such as methotrexate; an antiemetic drug such as aprepitant, dolasetron, fosaprepitant, granisetron, ondansetron, metoclopromide, hycosine or promethazine; a cardiovascular agent such as atenolol, dobutamine or epoprostenol; an anesthetic such as methohexital; and their pharmaceutically acceptable salts.

A combination of two or more drugs selected from small molecules, proteins, peptides and the like are also encompassed within the scope of the present invention.

As used herein, the term “absolute alcohol” refers to ethanol containing from about 98.0 to 99.8 v/v/ % of ethanol and from about 0.2 to 2.0 v/v % of water.

Within the context of the present invention and as used herein the term “subject” refers to an animal, preferably a mammal, and most preferably a human. In the context of the present invention, the term “mammal” is used interchangeably with the term “patient” or “subject”. In the context of the present invention the phrase “a subject in need thereof” means a subject (patient) in need of the treatment of a disease or disorder for which the pharmaceutically active agent is suitably used.

### Injectable Composition:

As discussed herein above, the inventors of the present invention have done extensive research and conducted several experiments to develop a stable injectable composition of a pharmaceutically active agent or a pharmaceutically acceptable salt or a co-crystal thereof; which can be prepared in a solubilized and stable form suitable for ready-to-use injection.

Further, being a RTU composition, it has enhanced patient compliance and also provides a more stable, safe and effective composition when compared to currently marketed lyophilized compositions.

In respect of the injectable composition of pharmaceutically active agents or a pharmaceutically acceptable salt or a co-crystal thereof; of the present invention, there is no requirement of reconstituting the composition with water prior to its administration, thus eliminating tedious task of reconstitution step in aseptic area, thereby providing an easy-to-use injectable composition.

The injectable composition of the present invention can be used for a wide variety of pharmaceutically active agents including peptide drugs, protein drugs, small molecule drugs and other therapeutically active agents.

The non-aqueous injectable composition of the present invention can be widely used for the delivery of numerous pharmaceutically active agents that are hydrophilic in nature bur are unstable in aqueous environment. Such pharmaceutically active agents include, but are not limited to, peptide drugs, protein drugs and small molecule drugs.

Accordingly, in one aspect, the present invention relates to a non-aqueous, stable and ready-to-use injectable composition of a pharmaceutically active agent or a pharmaceutically acceptable salt or a co-crystal thereof; wherein the said injectable composition comprises:
(i) a pharmaceutically active agent or a pharmaceutically acceptable salt or a co-crystal thereof;
(ii) a non-aqueous solvent system consisting of a primary non-aqueous solvent, optionally one or more secondary non-aqueous co-solvent(s);
(iii) optionally a polyol;
(iv) optionally a pH adjusting agent; and
(v) optionally an antioxidant.

In an embodiment, the pharmaceutically active agent is selected from a peptide drug, a protein drug or a small molecule drug.

In an embodiment, the pharmaceutically active agent is a peptide drug.

In an embodiment, the peptide drug is selected from calcitonin, leptin, melatonin, nafarelin, leuprolide, interferon-alpha, interferon-beta, interferon-gamma, low molecular weight heparin, imitrex, integrelin, nesiritide, nemifitide, sandostatin, cetrorelix acetate, ganirelix acetate, sermorelin acetate, zafirlukast, exanitide, pramlintide acetate, vasopressin, desmopressin, glucagon, oxytocin, corticorelin ovine triflutate, corticotropin releasing hormone, daptomycin, tobramycin, triptorelin, goserelin, fuzeon, hematide, buserelin, octreotide, gonadorelin, felypressin, deslorelin, vasopressin, eptifibatide, interleukin11, endostatin, angiostatin, N-acetyl oxyntomodulin 30-37, oxyntomodulin, ularitide, human Corticotropin-Releasing Factor (hCRF or Xerecept®), secretin, thymopentin, neuromedin U, neurotensin, elcatonin acetate, antide acetate, dynorphin A (1-13), acetate, sincalide, thymopentin acetate, thymosin alphal acetate (thymalfasin), fertirelin acetate, hisrelin, thymalfasin, ecallantide, oxycortin, urocortin, arixtra, urocortin, amylin, melanotan or valpreotide.

In an embodiment, the peptide drug is selected from daptomycin, nesiritide or cetrorelix acetate.

In an embodiment, the pharmaceutically active agent is a protein drug.

In an embodiment, the protein drug is a simple protein or a conjugated protein.

In an embodiment, the protein drug includes, but is not limited to, an enzyme, hormone or an antibody.

In an embodiment, the protein drug is an enzyme selected from: urokinase, streptokinase, kallikrein, pancreatic RNAase, platelet activating factor acetyl hydrolase, tissue plasminogen activator (TPA) or Superoxide dismutase (SOD).

In an embodiment, the protein drug is a hormone selected from insulin, gastrin prolactin, adrenocorticotropic hormone (ACTH), growth hormone (GH), thrombopoietin, obesity protein (leptin), Granulocyte colony-stimulating factor (G-CSF), Fibroblast growth factors (FGF), Insulin-like growth factors (IGF), Macrophage colony stimulating factor (M-CSF), Thyroid stimulating hormone (TSH), Luteinizing hormone (LH), Follicle stimulating hormone (FSH), Human chorionic gonadotropin (HCG) or Vascular endothelial growth factor (VEGF).

In an embodiment, the protein drug is a therapeutic agent that provides protection against diseases or other conditions, referred to as protection drug, which is selected from Osteoprotegerin (OPG), Alpha interferon, Beta interferon, Gamma interferon, Interleukin 2, Granulocyte macrophage colony stimulating factor (GM-CSF), Coagulation Factor IX, Tumor necrosis factor (TNF), Factor VII, Factor VIII, Factor IX, Colony stimulating growth factors (CSFs), Macrophage colony stimulating factor (M-CSF), Neurotrophic growth factor (NGF) or tumor necrosis factor binding protein (TNFbp).

In an embodiment, the protein drug is selected from kerantinocyte growth factor (KGF), Platelet-derived growth factor (PDGF), Bone morphogenetic protein (BMP) or Stem cell factor (SCF).

In an embodiment, the protein drug is selected from urokinase, streptokinase or prolactin.

In an embodiment, the pharmaceutically active agent is a small molecule drug. In an embodiment, the small molecule drug is a therapeutically active compound having molecular weight of less than about 3000 Daltons.

In an embodiment, the small molecule drug can be selected from the group of agents consisting of anti-cancer agents, anti-bacterial agents, immunomodulating agents, anti-obesity drugs, antidiabetic drugs, anti-fungal agents, anti-viral agents, contraceptives, analgesics, anti-inflammatory agents (e.g. steroids or non-steroidal anti-inflammatory drugs (NSAIDs)), antiemetic drugs, vasodilating agents, vasoconstricting agents, and cardiovascular agents.

In an embodiment, the small molecule drug is an anticancer agent selected from: azacitidine, bendamustine hydrochloride, bortezomib, carmustine, cisplatin, carboplatin, cyclophosphide, carmustine, daunorubicin hydrochloride, doxorubicin hydrochloride, etoposide, fludarabine, gemcitabine, melphalan, mitomycin, oxaliplatin, pemetrexed disodium, pentostatin, streptozocin, thiotepa, topotecan or vinblastine.

In an embodiment, the small molecule drug is a cytoprotective agent such as amifostine.

In an embodiment, the small molecule drug is an anti-bacterial agent selected from: tigecycline, doxycycline hyclate, chloramphenicol, azhithromycin or cefazolin sodium.

In an embodiment, the small molecule drug is an anti-fungal agent selected from: caspofungin, micafungin, anidulafungin or voriconazole.

In an embodiment, the small molecule drug is an anti-viral agent selected from acyclovir sodium or ganciclovir sodium.

In an embodiment, the small molecule drug is an anti-psychotic drug selected from thiothixene hydrochloride or midazolam hydrochloride.

In an embodiment, the small molecule drug is an anti-ulcer agent selected from esomeprazole sodium, lansoprazole or pantoprazole sodium.

In an embodiment, the small molecule drug is an analgesic selected from metamizole sodium, hydromorphone hydrochloride or remifentanil hydrochloride.

In an embodiment, the small molecule drug is an anti-inflammatory agent selected from hydrocortisone sodium succinate, methylprednisolone sodium succinate, indomethacin, ketoprofen or parecoxib sodium.

In an embodiment, the small molecule drug is an antiemetic drug selected from aprepitant, dolasetron mesylate, fosaprepitant, granisetron, ondansetron, metoclopromide hydrochloride, hycosine hydrobromide or promethazine.

In an embodiment, the small molecule drug is an immunomodulating agent such as methotrexate.

In an embodiment, the small molecule drug is a cardiovascular agent selected from atenolol, dobutamine hydrochloride or epoprostenol sodium.

In an embodiment, the small molecule drug is an anesthetic such as methohexital sodium.

In an embodiment, the small molecule drug is selected from caspofungin, pemetrexed, bortezomib or tigecycline.

In an embodiment, the pharmaceutically active agent is selected from: daptomycin, nesiritide, cetrorelix acetate, urokinase, streptokinase, prostacyclin, pemetrexed, bortezomib or tigecycline.

In an embodiment, the injectable composition contains the pharmaceutically active agent at a concentration in the range of about 0.1 mg/mL to about 250 mg/mL.

In an embodiment, the injectable composition contains the pharmaceutically active agent at a concentration in the range of about 0.1 mg/mL to about 100 mg/mL.

In an embodiment, the injectable composition contains the pharmaceutically active agent at a concentration in the range of about 0.1 mg/mL to about 50 mg/mL.

In an embodiment, the injectable composition contains the pharmaceutically active agent at a concentration in the range of about 0.1 mg/mL to about 20 mg/mL.

In an embodiment, the injectable composition contains the pharmaceutically active agent at a concentration in the range of about 0.1 mg/mL to about 10 mg/mL.

In an embodiment, the non-aqueous solvent system comprises 100% primary non-aqueous solvent; or in the non-aqueous solvent system, the primary non-aqueous solvent and the secondary non-aqueous co-solvent can be used in a ratio ranging from about 99:1 to about 50:50.

In an embodiment, the non-aqueous solvent system comprises 100% primary non-aqueous solvent.

In an embodiment, in the non-aqueous solvent system, the primary non-aqueous solvent and the secondary non-aqueous co-solvent can be used in the ratio ranging from about 99:1 to about 50:50.

In an embodiment, in the non-aqueous solvent system, the primary non-aqueous solvent and the secondary non-aqueous co-solvent can be used in the ratio of 99:1, 95:5, 90:10, 85:15, 80:20, 70:30, 60:40 or 50:50.

In an embodiment, in the non-aqueous solvent system, the primary non-aqueous solvent and the secondary non-aqueous co-solvent can be used in the ratio of 90:10.

In an embodiment, in the non-aqueous solvent system, the primary non-aqueous solvent and the secondary non-aqueous co-solvent can be used in the ratio of 85:15.

In an embodiment, the non-aqueous solvent system comprises one or more solvent(s) selected from the group consisting of but not limited to ethylene glycol, propylene glycol, glycerol, polyethylene glycol, dipropylene glycol, tripropylene glycol, methanol, ethanol, absolute alcohol, 1-propanol and isopropanol (isopropyl alcohol) or a mixture thereof.

In an embodiment, the primary non-aqueous solvent contained in the non-aqueous solvent system is selected from the group consisting of but not limited to ethylene glycol, propylene glycol, dipropylene glycol, tripropylene glycol, glycerol and polyethylene glycol or a mixture thereof.

In an embodiment, the primary non-aqueous solvent is propylene glycol.

In another embodiment, the optional secondary non-aqueous co-solvent contained in the non-aqueous solvent system is a (C₁-C₃)alkyl alcohol selected from the group consisting of but not limited to methanol, ethanol, absolute alcohol, 1-propanol and isopropanol (isopropyl alcohol) or a mixture thereof.

In another embodiment, the optional secondary non-aqueous co-solvent contained in the non-aqueous solvent system is isopropyl alcohol, ethanol or absolute alcohol; or a combination thereof.

In an embodiment, the optional secondary non-aqueous co-solvent(s) contained in the non-aqueous solvent system is ethanol or absolute alcohol.

In another embodiment, the optional secondary non-aqueous co-solvent contained in the non-aqueous solvent system is ethanol.

In another embodiment, the optional secondary non-aqueous co-solvent contained in the non-aqueous solvent system is absolute alcohol.

In another embodiment, the optional secondary non-aqueous co-solvent contained in the non-aqueous solvent system is isopropyl alcohol.

In another embodiment, the optional secondary non-aqueous co-solvent(s) contained in the non-aqueous solvent system is a combination of ethanol/absolute alcohol and isopropyl alcohol.

In an embodiment, the polyol is selected from a group consisting of but not limited to glycerin, sucrose, lactose, glucose, fructose, arabinose, xylose, ribose, mannose, galactose, dextrose, sorbose, sorbitol, mannitol, maltose, cellobiose, xylitol, or a combination thereof.

In an embodiment, the polyol is in the range of about 0.01% to about 10% of the total injectable composition of the pharmaceutically active agent.

In an embodiment, the polyol is sorbitol or racemic salts or isomers thereof.

In an embodiment, the polyol is D-sorbitol.

In the injectable composition of a pharmaceutically active agent or a pharmaceutically acceptable salt or a co-crystal thereof, the primary non-aqueous solvent, the optional secondary non-aqueous co-solvent and the optional polyol are present in an amount such that the pharmaceutically active agent is at a suitable concentration so that the pharmaceutically active agent is completely soluble and stable in the injectable composition.

In an embodiment, the non-aqueous solvent system contains propylene glycol and ethanol.

In an embodiment, the non-aqueous solvent system comprises 100% propylene glycol; or in the non-aqueous solvent system, the propylene glycol and the ethanol/absolute alcohol (and/or isopropyl alcohol) can be used in a ratio ranging from about 99:1 to about 50:50.

In an embodiment, the non-aqueous solvent system comprises 100% propylene glycol.

In an embodiment, in the non-aqueous solvent system, the propylene glycol and the ethanol/absolute alcohol (and/or isopropyl alcohol) can be used in the ratio ranging from about 99:1 to about 50:50.

In an embodiment, in the non-aqueous solvent system, the propylene glycol and the ethanol/absolute alcohol (and/or isopropyl alcohol) can be used in the ratio of 99:1, 95:5, 90:10, 85:15, 80:20, 70:30, 60:40 or 50:50.

In an embodiment, in the non-aqueous solvent system, the propylene glycol and ethanol/absolute alcohol (and/or isopropyl alcohol) can be used in the ratio of 90:10.

In an embodiment, in the non-aqueous solvent system, the propylene glycol and ethanol/absolute alcohol (and/or isopropyl alcohol) can be used in the ratio of 85:15.

In another embodiment, the pH adjusting agent is selected from pharmaceutically acceptable acids, bases, or buffering agents.

In another embodiment, the pH of the ready-to-use injectable composition of pharmaceutically active agent of the present invention is between about 2.0 and about 13.0.

In another embodiment, the pH of the ready-to-use injectable composition of pharmaceutically active agent of the present invention is between about 3.0 and about 13.0.

In an embodiment, the antioxidants may be selected from butylated hydroxytoluene, sodium metabisulphite acetylcysteine, ascorbyl palmitate, butylated hydroxyanisole, monothioglycerol, potassium nitrate, ascorbic acid or sodium ascorbate, sodium formaldehyde sulfoxylate, sodium bisulfite, vitamin E or a derivative thereof, propyl gallate, edetate, diethylenetriaminepentaacetic acid, triglycollamate, DL- or D-α-tocopherol, DL- or D-α-tocopheryl acetate, amino acids, stereoisomers of amino acids; or a combination thereof.

In another embodiment, the antioxidant may be selected from butylated hydroxytoluene or sodium metabisulphite.

### Process for the Preparation of Injectable Composition:

In an aspect, the present invention relates to a process for the preparation of a stable, non-aqueous and ready-to-use injectable composition of a pharmaceutically active ingredient or a pharmaceutically acceptable salt or a co-crystal thereof; wherein the said process comprises the steps of:
a) dissolving a pH adjusting agent in a non-aqueous solvent system consisting of a primary non-aqueous solvent to obtain a first solution by stirring the solution at a temperature ranging from 2° C. to 60° C. over a period of 30 minutes to 120 minutes and allowing the solution to attain the temperature of 2° C. to room temperature;
b) optionally adding polyol and antioxidant to the secondary non-aqueous co-solvent under constant stirring until the polyol dissolves to obtain a second solution;
c) adding the second solution of step (b) to the first solution of step (a) under constant stirring to obtain a third solution;
d) dispersing the pharmaceutically active ingredient in the third solution of step (c) to obtain a clear solution;
e) optionally filtering the clear solution of step (d); and
f) filling the clear solution of step (e) into a container to obtain a preparation in a ready-to-use form.

In an embodiment, the present invention relates to a process for the preparation of a stable, non-aqueous and ready-to-use injectable composition of a pharmaceutically active ingredient or a pharmaceutically acceptable salt or a co-crystal thereof; wherein the said process comprises the steps of:
a) dissolving polyol, optionally antioxidant and pH adjusting agent in secondary non-aqueous co-solvent to obtain a first solution by stirring the solution at a temperature ranging from 2° C. to 60° C. over a period of 30 minutes to 120 minutes and allowing the solution to attain the temperature of 2° C. to room temperature;
b) adding primary non-aqueous solvent to the first solution of step (a) to obtain a second solution;
c) adding pharmaceutically active ingredient to the second solution of step (b) and allowing to disperse to produce a solution;
d) optionally filtering the solution of step (c) one or more times to obtain a clear solution; and
e) filling the clear solution of step (d) into a container to obtain a composition in a ready-to-use form.

In an embodiment, the present invention relates to a process for the preparation of the stable, non-aqueous and ready-to-use injectable composition of a pharmaceutically active ingredient or a pharmaceutically acceptable salt or a co-crystal thereof; wherein the said process comprises the steps of:
a) dissolving a pH adjusting agent in a non-aqueous solvent system consisting of a primary non-aqueous solvent to obtain a mixture and stirring the resulting mixture at a temperature ranging from 2° C. to 60° C. over a period of 30 minutes to 120 minutes to obtain a first solution;
b) allowing the resulting first solution of step (a) to attain a temperature of 2° C. to room temperature;
c) optionally adding polyol and antioxidant to the first solution of step (b) under constant stirring until the polyol dissolves, to obtain a second solution;
d) optionally adding a secondary non-aqueous co-solvent to the second solution of step (c) under constant stirring for 5 minutes to 10 minutes to obtain a third solution;
e) adding pharmaceutically active ingredient to the third solution of step (d) and allowing to disperse to obtain a solution;
f) optionally filtering the solution as obtained in step (e) one or more times to obtain a clear solution; and
g) filling the clear solution of step (f) in suitable containers to obtain a composition in a ready-to-use form.

In an embodiment, the present invention relates to a process for the preparation of the stable, non-aqueous and ready-to-use injectable composition of a pharmaceutically active ingredient or a pharmaceutically acceptable salt or a co-crystal thereof; wherein the said process comprises the steps of:
a) dissolving polyol, optionally antioxidant and pH adjusting agent in primary non-aqueous solvent to obtain a solution by heating the resulting mixture at a temperature ranging from 2° C. to 60° C. over a period of 30 minutes to 120 minutes to obtain a first solution;
b) allowing the resulting first solution of step (a) to attain a temperature of 2° C. to room temperature;
c) optionally adding a secondary non-aqueous solvent to the first solution of step (b) under constant stirring for 5 minutes to 10 minutes to obtain a second solution; d) adding pharmaceutically active ingredient to the second solution of step (c) and allowing to disperse to obtain a solution;
e) optionally filtering the solution of step (d) one or more times to obtain a clear solution; and
f) filling the clear solution of step (e) in suitable containers to obtain a composition in a ready-to-use form.

In an embodiment, in the process for the preparation of the injectable composition of the pharmaceutically active agent; the said pharmaceutically active agent is as described above in one or more embodiments of the invention.

In an embodiment, in the process for the preparation of the injectable composition of a pharmaceutically active agent or a pharmaceutically acceptable salt or a co-crystal thereof; the non-aqueous solvent system comprises 100% primary non-aqueous solvent; or in the non-aqueous solvent system, the primary non-aqueous solvent and the secondary non-aqueous co-solvent can be used in a ratio ranging from about 99:1 to about 50:50.

In an embodiment, in the process for the preparation of the injectable composition of a pharmaceutically active agent or a pharmaceutically acceptable salt or a co-crystal thereof; the non-aqueous solvent system comprises 100% primary non-aqueous solvent.

In an embodiment, in the process for the preparation of the injectable composition of a pharmaceutically active agent or a pharmaceutically acceptable salt or a co-crystal thereof; in the non-aqueous solvent system, the primary non-aqueous solvent and the secondary non-aqueous co-solvent can be used in the ratio ranging from about 99:1 to about 50:50.

In an embodiment, in the process for the preparation of the injectable composition of a pharmaceutically active agent or a pharmaceutically acceptable salt or a co-crystal thereof; in the non-aqueous solvent system, the primary non-aqueous solvent and the secondary non-aqueous co-solvent can be used in the ratio of 99:1, 95:5, 90:10, 85:15, 80:20, 70:30, 60:40 or 50:50.

In an embodiment, in the process for the preparation of the injectable composition of a pharmaceutically active agent or a pharmaceutically acceptable salt or a co-crystal thereof; in the non-aqueous solvent system, the primary non-aqueous solvent and the secondary non-aqueous co-solvent(s) can be used in the ratio of 90:10.

In an embodiment, in the process for the preparation of the injectable composition of a pharmaceutically active agent or a pharmaceutically acceptable salt or a co-crystal thereof; in the non-aqueous solvent system, the primary non-aqueous solvent and the secondary non-aqueous co-solvent(s) can be used in the ratio of 85:15.

In an embodiment, in the process for the preparation of the injectable composition of the pharmaceutically active agent or a pharmaceutically acceptable salt or a co-crystal thereof; the primary non-aqueous solvent contained in the non-aqueous solvent system is selected from the group consisting of but not limited to ethylene glycol, propylene glycol, dipropylene glycol, tripropylene glycol, glycerol and polyethylene glycol or a mixture thereof.

In an embodiment, in the process for the preparation of the injectable composition of the pharmaceutically active agent or a pharmaceutically acceptable salt or a co-crystal thereof; the primary non-aqueous solvent is propylene glycol.

In another embodiment, in the process for the preparation of the injectable composition of the pharmaceutically active agent or a pharmaceutically acceptable salt or a co-crystal thereof; the secondary non-aqueous co-solvent is a (C₁-C₃)alkyl alcohol selected from the group consisting of but not limited to methanol, ethanol, absolute alcohol, 1-propanol and isopropanol (isopropyl alcohol) or a mixture thereof.

In another embodiment, in the process for the preparation of the injectable composition of the pharmaceutically active agent or a pharmaceutically acceptable salt or a co-crystal thereof; the secondary non-aqueous co-solvent is isopropyl alcohol; ethanol or absolute alcohol; or a combination thereof.

In another embodiment, in the process for the preparation of the injectable composition of the pharmaceutically active agent or a pharmaceutically acceptable salt or a co-crystal thereof; the secondary non-aqueous co-solvent is ethanol.

In another embodiment, in the process for the preparation of the injectable composition of the pharmaceutically active agent or a pharmaceutically acceptable salt or a co-crystal thereof; the secondary non-aqueous co-solvent is absolute alcohol.

In another embodiment, in the process for the preparation of the injectable composition of the pharmaceutically active agent or a pharmaceutically acceptable salt or a co-crystal thereof; the secondary non-aqueous co-solvent is isopropyl alcohol.

In another embodiment, in the process for the preparation of the injectable composition of the pharmaceutically active agent or a pharmaceutically acceptable salt or a co-crystal thereof; the secondary non-aqueous co-solvent is a combination of ethanol/absolute alcohol and isopropyl alcohol.

In an embodiment, in the process for the preparation of the injectable composition of the pharmaceutically active agent or a pharmaceutically acceptable salt or a co-crystal thereof; the polyol is selected from the group consisting of but not limited to glycerin, sucrose, lactose, glucose, fructose, arabinose, xylose, ribose, mannose, galactose, dextrose, sorbose, sorbitol, mannitol, maltose, cellobiose, xylitol, or a combination thereof.

In an embodiment, in the process for the preparation of the injectable composition of the pharmaceutically active agent or a pharmaceutically acceptable salt or a co-crystal thereof; the polyol is in the range of about 0.01% to about 10% of the total injectable composition of the pharmaceutically active agent.

In an embodiment, in the process for the preparation of the injectable composition of the pharmaceutically active agent or a pharmaceutically acceptable salt or a co-crystal thereof; the polyol is sorbitol or racemic salts or isomers thereof.

In an embodiment, in the process for the preparation of the injectable composition of the pharmaceutically active agent or a pharmaceutically acceptable salt or a co-crystal thereof; the polyol is D-sorbitol.

In an embodiment, in the process for the preparation of the injectable composition of a pharmaceutically active agent or a pharmaceutically acceptable salt or a co-crystal thereof; the non-aqueous solvent system comprises 100% propylene glycol.

In an embodiment, in the process for the preparation of the injectable composition of the pharmaceutically active agent or a pharmaceutically acceptable salt or a co-crystal thereof; the non-aqueous solvent system comprises propylene glycol and ethanol.

In an embodiment, in the process for the preparation of the injectable composition of the pharmaceutically active agent or a pharmaceutically acceptable salt or a co-crystal thereof; the non-aqueous solvent system comprises propylene glycol and absolute alcohol.

In an embodiment, in the process for the preparation of the injectable composition of the pharmaceutically active agent or a pharmaceutically acceptable salt or a co-crystal thereof; the non-aqueous solvent system comprises propylene glycol and isopropyl alcohol.

In an embodiment, in the process for the preparation of the injectable composition of the pharmaceutically active agent or a pharmaceutically acceptable salt or a co-crystal thereof; the non-aqueous solvent system comprises 100% propylene glycol; or in the non-aqueous solvent system, the propylene glycol and ethanol/absolute alcohol (and/or isopropyl alcohol) can be used in a ratio ranging from about 99:1 to about 50:50.

In an embodiment, in the process for the preparation of the injectable composition of the pharmaceutically active agent or a pharmaceutically acceptable salt or a co-crystal thereof; the non-aqueous solvent system comprises 100% propylene glycol.

In an embodiment, in the process for the preparation of the injectable composition of the pharmaceutically active agent or a pharmaceutically acceptable salt or a co-crystal thereof; in the non-aqueous solvent system, the propylene glycol and ethanol/absolute alcohol (and/or isopropyl alcohol) can be used in the ratio ranging from about 99:1 to about 50:50.

In an embodiment, in the process for the preparation of the injectable composition of the pharmaceutically active agent; in the non-aqueous solvent system, propylene glycol and ethanol/absolute alcohol (and/or isopropyl alcohol) can be used in the ratio of 99:1, 95:5, 90:10, 85:15, 80:20, 70:30, 60:40 or 50:50.

In an embodiment, in the process for the preparation of the injectable composition of the pharmaceutically active agent or a pharmaceutically acceptable salt or a co-crystal thereof; in the non-aqueous solvent system, propylene glycol and ethanol/absolute alcohol (and/or isopropyl alcohol) can be used in the ratio of 90:10.

In an embodiment, in the process for the preparation of the injectable composition of the pharmaceutically active agent or a pharmaceutically acceptable salt or a co-crystal thereof; in the non-aqueous solvent system, propylene glycol and ethanol/absolute alcohol (and/or isopropyl alcohol) can be used in the ratio of 85:15.

In an embodiment, in the process for the preparation of the injectable composition of the pharmaceutically active agent or a pharmaceutically acceptable salt or a co-crystal thereof; the pH adjusting agent is selected from pharmaceutically acceptable acids, bases, or buffering agents.

In another embodiment, the pH of the ready-to-use injectable composition of the pharmaceutically active agent or a pharmaceutically acceptable salt or a co-crystal thereof; obtained by the process as described above is between about 2.0 and about 13.0.

In another embodiment, the pH of the ready-to-use injectable composition of the pharmaceutically active agent or a pharmaceutically acceptable salt or a co-crystal thereof; obtained by the process as described above is between about 3.0 and about 13.0.

In an embodiment, in the process for the preparation of the injectable composition of a peptide drug or a pharmaceutically acceptable salt or a co-crystal thereof; the antioxidant is selected from but not limited to butylated hydroxytoluene, sodium metabisulphite acetylcysteine, ascorbyl palmitate, butylated hydroxyanisole, monothioglycerol, potassium nitrate, ascorbic acid or sodium ascorbate, sodium formaldehyde sulfoxylate, sodium bisulfite, vitamin E or a derivative thereof, propyl gallate, edetate, diethylenetriaminepentaacetic acid, triglycollamate, DL- or D-α-tocopherol, DL- or D-α-tocopheryl acetate, amino acids, stereoisomers of amino acids; or a combination thereof.

In an embodiment, in the process for the preparation of the injectable composition of a peptide drug or a pharmaceutically acceptable salt or a co-crystal thereof; the antioxidant is selected from butylated hydroxytoluene or sodium metabisulphite.

### Method of Use of the Injectable Composition:

In an aspect, the present invention relates to use of a stable, non-aqueous and ready-to-use injectable composition of the pharmaceutically active agent or a pharmaceutically acceptable salt or a co-crystal thereof; for the manufacture of a medicament for treating or preventing one or more diseases, conditions or disorders; wherein the said injectable composition is as described in one or more embodiments of the present invention as described herein above.

In another embodiment, the present invention relates to a method of treating or preventing one or more diseases, conditions or disorders comprising administering to a subject in need thereof a therapeutically effective amount of a stable, non-aqueous and ready-to-use injectable composition of the pharmaceutically active agent or a pharmaceutically acceptable salt or a co-crystal thereof; wherein the said injectable composition is as described in one or more embodiments of the present invention as described herein above.

In an embodiment, the diseases, disorders or conditions for the treatment or prevention of which the injectable composition of a pharmaceutically active agent or a pharmaceutically acceptable salt or a co-crystal thereof; of the present invention can be used, include, but are not limited to, metabolic disorders, autoimmune disorders, cardiovascular diseases, respiratory diseases, thyroid diseases, hormonal diseases, neurodegenerative diseases, bacterial infections, viral infections, fungal infections, renal diseases, hepatobiliary diseases, venereal diseases, platelet aggregation, inflammatory diseases, cancers, transplantation complications due to rejection reactions, graft rejection and hepatic diseases.

In an embodiment, the pharmaceutically active agent contained in the stable, non-aqueous and ready-to-use injectable composition which is provided for use in the treatment or prevention of one or more diseases, conditions or disorders (as described herein); is as described above in one or more embodiments of the invention.

In another embodiment, the stable, non-aqueous and ready-to-use injectable composition of a pharmaceutically active agent or a pharmaceutically acceptable salt or a co-crystal thereof; can be packaged in a suitable container depending upon the composition and the method of administration of the composition. Suitable containers known to a person skilled in the art include vials, ampoules and infusion bag.

In another embodiment, the present invention provides a pharmaceutical kit comprising the stable, non-aqueous and ready-to-use injectable composition of a pharmaceutically active agent; wherein the said composition comprises of the pharmaceutically active agent or a pharmaceutically acceptable salt or a co-crystal thereof; a non-aqueous solvent system consisting of a primary non-aqueous solvent, optionally one or more secondary non-aqueous co-solvent(s); optionally a polyol; optionally a pH adjusting agent and optionally an antioxidant. The kit may further comprise a package insert, including information about the indication, usage, doses, direction for administration, contraindications, precautions and warnings. The kit may further contain optional materials for storing and/or administering the drug like infusion bag as well as instructions for storage and use.

In another embodiment, the stable, non-aqueous and ready-to-use injectable composition of a pharmaceutically active agent or a pharmaceutically acceptable salt or a co-crystal thereof; of the present invention can be delivered to the subject intravenously. Methods of delivering the RTU injectable composition of a pharmaceutically active agent intravenously are well known in the art.

In another embodiment, the stable, non-aqueous and ready-to-use injectable composition of a pharmaceutically active agent or a pharmaceutically acceptable salt or a co-crystal thereof; of the present invention can be delivered to the subject by infusion. For example, the injectable dosage form may be delivered intravenously through infusion.

It is understood that modifications that do not substantially affect the activity of the various embodiments of this invention are included within scope of the invention disclosed herein. Accordingly, the following examples are intended to illustrate but not to limit the scope of the present invention.

### EXAMPLES

### Example 1

### The Injectable Composition of Daptomycin (RTU)

| Ingredients | mg/mL |
|---|---|
| Daptomycin | 50 |
| Propylene Glycol | 988 |
| Ethanol | 39.45 |
| D-Sorbitol | 0.1093 |
| Sodium Hydroxide | q.s. (quantity sufficient) |

Procedure:
a) Propylene glycol was taken in a glass bottle/stainless steel (SS) container.
b) Sodium hydroxide was dissolved in propylene glycol of step (a) to obtain a first solution by stirring for 60 minutes and attaining temperature of 2° C. to 8° C.
c) Sorbitol was dissolved in ethanol to obtain a second solution.
d) The second solution obtained in step (c) was added to first solution obtained in step (b).
e) Daptomycin was then added to the solution obtained in step (d) to obtain a solution.
f) The solution obtained in step (e) was subjected to turbulence for 30-120 minutes to obtain a clear solution.
g) The clear liquid concentrate obtained in step (f) was filled in siliconised/non-siliconised vial and stoppered with Teflon coated rubber stoppers with nitrogen headspace to obtain a composition in a ready-to-use form.

### Example 2

### The Injectable Composition of Daptomycin (RTU)

| Ingredients | mg/mL |
|---|---|
| Daptomycin | 50 |
| Propylene Glycol | 988 |
| Ethanol | 39.45 |
| D-Sorbitol | 0.1093 |

### Procedure:

a) Propylene glycol was taken in a glass bottle/stainless steel (SS) container.
b) Sodium hydroxide was dissolved in propylene glycol of step (a) to obtain a first solution by stirring for 60 minutes and attaining temperature of 2° C. to 8° C.
c) Sorbitol was dissolved in ethanol to obtain a second solution.
d) The second solution obtained in step (c) was added to first solution obtained in step (b).
e) Daptomycin was then added to the solution obtained in step (d) to obtain a solution.
f) The solution obtained in step (e) was subjected to turbulence for 30-120 minutes to obtain a clear solution.
g) The clear liquid concentrate obtained in step (f) was filled in siliconised/non-siliconised vial and stoppered with Teflon coated rubber stoppers with nitrogen headspace to obtain a composition in a ready-to-use form.

### Stability Studies (Example 2):

| | | Storage conditions | | | | | |
|---|---|---|---|---|---|---|---|
| | | 2-8° C. | | 15° C. | | 25° C./60% RH | |
| Parameters | Initial | 1 M | 2 M | 1 M | 2 M | 1 M | 2 M |
| Assay | 97.9 | 96.0 | 97.6 | 90.6 | 90.1 | 82.5 | 74.0 |

M—Months

Results of the stability studies performed for ready to use injectable Daptomycin composition mentioned according to Example 2 demonstrates that 1.9% degradation occurred at 2-8° C. after one month stability whereas the composition was stable without degradation after 2 months. The composition is stable at 2-8° C.

### Example 3

### The Injectable Composition of Daptomycin (RTU)

| Ingredients | mg/mL |
|---|---|
| Daptomycin | 50 |
| Propylene Glycol | 988 |
| Ethanol | 39.45 |
| D-Sorbitol | 0.1093 |
| Butylated hydroxytoluene | 0.02 |

### Procedure:

a) Propylene glycol was taken in a glass bottle/SS container.
b) Sorbitol and butylated hydroxytoluene were dissolved in ethanol to obtain a solution.
c) The solution thus obtained in step (b) was added to propylene glycol of step (a) with continuous stirring until complete miscibility was observed.
d) Daptomycin was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Stability Studies (Example 3):

Storage conditions

| | | 2-8° C. | 15° C. | 25° C./60% RH |
|---|---|---|---|---|
| Parameters | Initial | 1 M | 1 M | 1 M |
| Assay | 102.7 | 100.9 | 97.1 | 88.7 |

M—Months

Results of the stability studies performed for ready to use injectable Daptomycin composition mentioned according to Example 3 demonstrates that the composition exhibited stability upto 1 month at 2-8° C.

### Example 4

### The Injectable Composition of Daptomycin (RTU)

| Ingredients | mg/mL |
|---|---|
| Daptomycin | 50 |
| Propylene Glycol | 988 |
| Ethanol | 39.45 |
| D-Sorbitol | 0.1093 |
| Sodium Metabisulphite | 0.50 |

### Procedure:

a) Propylene glycol was taken in a glass bottle/SS container.
b) Sorbitol and sodium metabisulphite were dissolved in ethanol to obtain a solution.
c) The solution thus obtained in step (b) was added to propylene glycol of step (a) with continuous stirring until complete miscibility was observed.
d) Daptomycin was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form

### Stability Studies (Example 4):

Storage conditions

| | | 2-8° C. | 15° C. | 25° C./60% RH |
|---|---|---|---|---|
| Parameters | Initial | 1 M | 1 M | 1 M |
| Assay | 103.9 | 99.5 | 92.8 | 91.6 |

M—Months

Results of the stability studies performed for ready to use injectable Daptomycin composition mentioned according to Example 4 demonstrates that the composition exhibited stability upto 1 month at 2-8° C.

### Example 5

### The Injectable Composition of Urokinase (RTU)

| Ingredients | mg/mL |
|---|---|
| Urokinase | 1000000 IU |
| Propylene Glycol | 988 |
| Ethanol | 39.45 |
| D-Sorbitol | 0.1093 |
| Sodium Hydroxide | q.s. (quantity sufficient) |

Procedure:
a) Sorbitol was dissolved in ethanol to obtain a solution.
b) Propylene glycol was then dissolved in the solution obtained in the step (a).
c) Sodium hydroxide was dissolved in a solution obtained in the step (b) by heating at 50° C. for 60 minutes and cooling to a temperature of 2° C. to 8° C.
d) Urokinase was then added to the solution of step (c) to obtain another solution.
e) The solution obtained in step (d) was subjected to turbulence to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Example 6

### The Injectable Composition of Urokinase (RTU)

| Ingredients | mg/mL |
|---|---|
| Urokinase | 0.5 |
| Propylene Glycol | 936 |
| Ethanol | 78.9 |
| D-Sorbitol | 0.1093 |
| Sodium Hydroxide | 5.5 |

### Procedure:

a) Propylene glycol was taken in a glass bottle/SS container.
b) Sodium hydroxide was dissolved in a solution obtained in the step (a) by heating at 50° C. for 60 minutes and cooling to a temperature of 2° C. to 8° C.
c) Sorbitol was dissolved in ethanol to obtain a solution.
d) The solution obtained in step (c) was added to the solution obtained in (b).
e) Urokinase was then added to the solution obtained in step (d) to obtain a solution.
f) The solution obtained in step (e) was subjected to turbulence to obtain a clear solution.
g) The clear liquid concentrate obtained in step (f) is filled in siliconised/non-siliconised vial and stoppered with Teflon coated rubber stoppers with nitrogen headspace to obtain a composition in a ready-to-use form.

### Stability Studies (Example 6):

Storage conditions

| | | 2-8° C. | 15° C. |
|---|---|---|---|
| Parameters | Initial | 1 M | 1 M |
| Assay (%)* | 76.93 | 73.20 | 81.04 |

M—Months

Results of the stability studies performed for ready to use injectable urokinase composition mentioned according to Example 6 demonstrates that the composition exhibited stability upto 1 month at all storage conditions.

* Similar results were obtained using Bradford protein assay methods.

### Example 7

### The Injectable Composition of Caspofungin (RTU)

| Ingredients | mg/mL |
|---|---|
| Caspofungin | 7 |
| Propylene Glycol | 988 |
| Ethanol | 39.45 |
| D-Sorbitol | 0.1093 |
| Sodium Hydroxide | q.s. (quantity sufficient) |

Procedure:
a) Sodium hydroxide was added to propylene glycol to obtain a first solution by stirring for 60 minutes and cooling to a temperature of 2° C. to 8° C.
b) Sorbitol (was dissolved in ethanol to obtain a second solution.
c) The second solution of step (b) was added to the first solution of step (a) to obtain a third solution.
d) Caspofungin was then added to the solution obtained in step (c) to obtain another solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Example 8

### The Injectable Composition of Caspofungin (RTU)

| Ingredients | mg/mL |
|---|---|
| Caspofungin | 10 |
| Propylene Glycol | 988 |
| Ethanol | 39.45 |
| D-Sorbitol | 0.1093 |

Procedure:
a) Propylene glycol was taken in a glass bottle/stainless steel (SS) container.
b) Sorbitol was dissolved in ethanol to obtain a solution.
c) The solution thus obtained in step (b) was added to propylene glycol of step (a) with continuous stirring until complete miscibility was observed.
d) Caspofungin was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Stability Studies (Example 8):

| Storage conditions | | |
|---|---|---|
| | 2-8° C. | 15° C. |

| Parameters | Initial | 1 M | 2 M | 1 M |
|---|---|---|---|---|
| Assay | 96.7 | 95.4 | 97.2 | 87.5 |

M—Months

Results of the stability studies performed for ready to use injectable caspofungin composition mentioned according to Example 8 demonstrates that no degradation occurred at 2-8° C. The composition is stable at 2-8° C.

### Example 9

### The Injectable Composition of Caspofungin (RTU)

| Ingredients | mg/mL |
|---|---|
| Caspofungin | 10 |
| Propylene Glycol | 988 |
| Ethanol | 39.45 |
| D-Sorbitol | 0.1093 |
| Butylated hydroxytoluene | 0.02 |

Procedure:
a) Propylene glycol was taken in a glass bottle/SS container.
b) Sorbitol and butylated hydroxytoluene were dissolved in ethanol to obtain a solution.
c) The solution thus obtained in step (b) was added to propylene glycol of step (a) with continuous stirring until complete miscibility was observed.
d) Caspofungin was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Stability Studies (Example 9):

| Storage conditions | | |
|---|---|---|
| | 2-8° C. | 15° C. |

| Parameters | Initial | 1 M | 2 M | 1 M |
|---|---|---|---|---|
| Assay | 96.0 | 95.6 | 94.5 | 88.7 |

M—Months

Results of the stability studies performed for ready to use injectable caspofungin composition mentioned according to Example 9 demonstrates that no degradation occurred at 2-8° C. The composition exhibited stability at 2-8° C. upto 2 months.

### Example 10

### The Injectable Composition of Caspofungin (RTU)

| Ingredients | mg/mL |
|---|---|
| Caspofungin | 10 |
| Propylene Glycol | 988 |
| Ethanol | 39.45 |
| D-Sorbitol | 0.1093 |
| Sodium Metabisulphite | 0.50 |

### Procedure:

a) Propylene glycol was taken in a glass bottle/SS container.
b) Sorbitol and sodium metabisulphite were dissolved in ethanol to obtain a solution.
c) The solution thus obtained in step (b) was added to propylene glycol of step (a) with continuous stirring until complete miscibility was observed.
d) Caspofungin was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Stability Studies (Example 10):

| Storage conditions | | |
|---|---|---|
| | 2-8° C. | 15° C. |

| Parameters | Initial | 1 M | 2 M | 1 M |
|---|---|---|---|---|
| Assay | 96.2 | 96.4 | 94.4 | 88.7 |

M—Months

Results of the stability studies performed for ready to use injectable caspofungin composition mentioned according to Example 10 demonstrates that no degradation occurred at 2-8° C. The composition exhibited stability at 2-8° C. upto 2 months.

### Example 11

### The Injectable Composition of Fosaprepitant (RTU)

| Ingredients | mg/mL |
|---|---|
| Fosaprepitant | 30 |
| Propylene Glycol | 988 |
| Ethanol | 39.45 |
| D-Sorbitol | 0.1093 |

### Procedure:

a) Propylene glycol was taken in a glass bottle/SS container.
b) Sorbitol was dissolved in ethanol to obtain a solution.
c) The solution thus obtained in step (b) was added to propylene glycol of step (a) with continuous stirring until complete miscibility was observed.
d) Fosaprepitant was then added to the solution obtained in step (c) to obtain a solution.
e) The solution obtained in step (d) was subjected to turbulence for 30-120 minutes to obtain a clear solution.
f) The clear liquid concentrate obtained in step (e) was filled in siliconised/non-siliconised vial and stoppered with Teflon coated rubber stoppers with nitrogen headspace to obtain a composition in a ready-to-use form.

### Stability Studies (Example 11):

Storage conditions

| | | 2-8° C. | 15° C. |
|---|---|---|---|
| Parameters | Initial | 1 M | 1 M |
| Assay | 106.3 | 94.0 | 102.0 |

M—Months

Results of the stability studies performed for ready to use injectable fosaprepitant composition mentioned according to Example 11 demonstrates that the formulation exhibited stability upto 1 month at both the storage conditions.

### Example 12

### The Injectable Composition of Fosaprepitant (RTU)

| Ingredients | mg/mL |
|---|---|
| Fosaprepitant | 50 |
| Propylene Glycol | 988 |
| Ethanol | 39.45 |
| D-Sorbitol | 0.1093 |

The composition described in Example 12 is prepared by following the same procedure as described in the above Example 11.

### Stability Studies (Example 12):

Storage conditions

| | | 2-8° C. | 15° C. |
|---|---|---|---|
| Parameters | Initial | 1 M | 1 M |
| Assay | 106.0 | 99.0 | 98.3 |

M—Months

Results of the stability studies performed for ready to use injectable fosaprepitant composition mentioned according to Example 12 demonstrates that the formulation exhibited stability upto 1 month at both the storage conditions.

### Example 13

### The Injectable Composition of Fosaprepitant (RTU)

| Ingredients | mg/mL |
|---|---|
| Fosaprepitant | 30 |
| Propylene Glycol | 988 |
| Ethanol | 39.45 |
| D-Sorbitol | 0.1093 |
| Sodium Hydroxide (NaOH) | 4.32 |

### Procedure:

a) Propylene glycol was taken in a glass bottle/SS container.
b) Sorbitol was dissolved in ethanol to obtain a solution.
c) Propylene glycol was then dissolved in the solution obtained in the step (b).
d) Sodium hydroxide was dissolved in a solution obtained in the step (c) by heating at 50° C. for 60 minutes and cooling to a temperature of 2° C. to 8° C.
e) Fosaprepitant was then added to the solution to obtain a solution.
f) The solution obtained in step (e) was subjected to turbulence for 30-120 minutes to obtain a clear solution.
g) The clear liquid concentrate obtained in step (f) was filled in siliconised/non-siliconised vial and stoppered with Teflon coated rubber stoppers with nitrogen headspace to obtain a formulation in a ready-to-use form.

### Stability Studies (Example 13):

Storage conditions

| | | 2-8° C. | 15° C. |
|---|---|---|---|
| Parameters | Initial | 1 M | 1 M |
| Assay | 99.0 | 91.9 | 92.9 |

M—Months

Results of the stability studies performed for ready to use injectable fosaprepitant composition mentioned according to Example 13 demonstrates that the formulation exhibited stability upto 1 month at both the storage conditions.

### Example 14

### The Injectable Composition of Fosaprepitant (RTU)

| Ingredients | mg/mL |
|---|---|
| Fosaprepitant | 50 |
| Propylene Glycol | 988 |
| Ethanol | 39.45 |
| D-Sorbitol | 0.1093 |
| Sodium Hydroxide (NaOH) | 4.32 |

The composition described in Example 14 is prepared by following the same procedure as described in the above Example 13.

### Stability Studies (Example 14):

Storage conditions

| | | 2-8° C. | 15° C. |
|---|---|---|---|
| Parameters | Initial | 1 M | 1 M |
| Assay | 104.4 | 90.1 | 90.2 |

M—Months

Results of the stability studies performed for ready to use injectable fosaprepitant composition mentioned according to Example 14 demonstrates that the formulation exhibited stability upto 1 month at both the storage conditions.

1. A stable, non-aqueous and ready-to-use injectable composition of a pharmaceutically active agent or a pharmaceutically acceptable salt or a co-crystal thereof; comprising:
(i) a pharmaceutically active agent or a pharmaceutically acceptable salt or a co-crystal thereof;
(ii) a non-aqueous solvent system;
(iii) optionally a polyol;
(iv) optionally a pH adjusting agent; and
(v) optionally an antioxidant.

2. The injectable composition according to claim 1, wherein the pharmaceutically active agent is selected from a peptide drug, a protein drug or a small molecule drug or a bioconjugate; or a combination thereof.

3. The injectable composition according to claim 2, wherein the peptide drug is selected from daptomycin, nesiritide, cetrorelix acetate; or a combination thereof.

4. The injectable composition according to claim 2, wherein the protein drug is selected from urokinase, streptokinase, prolactin; or a combination thereof.

5. The injectable composition according to claim 2, wherein the small molecule drug is selected from caspofungin, pemetrexed, bortezomib, tigecycline, fosaprepitant; or a combination thereof.

6. The injectable composition according to claim 1, wherein the non-aqueous solvent system comprises of a primary non-aqueous solvent.

7. The injectable composition according to claim 1, wherein the non-aqueous solvent system comprises of a primary non-aqueous solvent and one or more secondary non-aqueous co-solvents.

8. The injectable composition according to claim 1, wherein the non-aqueous solvent system comprises one or more solvent selected from the group consisting of ethylene glycol, propylene glycol, dipropylene glycol, tripropylene glycol, glycerol, polyethylene glycol, methanol, ethanol, absolute alcohol, 1-propanol and isopropanol (isopropyl alcohol) or a mixture thereof.

9. The injectable composition according to claim 6, wherein the primary non-aqueous solvent contained in the non-aqueous solvent system is selected from the group consisting of ethylene glycol, propylene glycol, dipropylene glycol, tripropylene glycol, glycerol and polyethylene glycol or a mixture thereof.

10. The injectable composition according to claim 7, wherein the secondary non-aqueous co-solvent contained in the non-aqueous solvent system is a (C₁-C₃)alkyl alcohol selected from the group consisting of but not limited to methanol, ethanol, absolute alcohol, 1-propanol and isopropanol (isopropyl alcohol) or a mixture thereof.

11. The injectable composition according to claim 1, wherein the polyol is selected from a group consisting of glycerin, sucrose, lactose, glucose, fructose, arabinose, xylose, ribose, mannose, galactose, dextrose, sorbose, sorbitol, mannitol, maltose, cellobiose, xylitol, or a combination thereof.

12. The injectable composition according to claim 1, wherein the pH adjusting agent is selected from pharmaceutically acceptable acids, bases, or buffering agents.

13. The injectable composition according to claim 1, wherein the antioxidant is selected from butylated hydroxytoluene, sodium metabisulphite acetylcysteine, ascorbyl palmitate, butylated hydroxyanisole, monothioglycerol, potassium nitrate, ascorbic acid or sodium ascorbate, sodium formaldehyde sulfoxylate, sodium bisulfite, vitamin E or a derivative thereof, propyl gallate, edetate, diethylenetriaminepentaacetic acid, triglycollamate, DL- or D-α-tocopherol, DL- or D-α-tocopheryl acetate, amino acids, stereoisomers of amino acids; or a combination thereof.

14. The injectable composition according to claim 1, wherein the said pharmaceutically active agent is in the range of 0.1 mg/mL to 250 mg/mL.

15. The injectable composition according to claim 7, wherein the said injectable composition comprises the primary non-aqueous solvent and the secondary non-aqueous co-solvent in the ratio ranging from 99:1 to 50:50.

16. The injectable composition according to claim 1, wherein the polyol is in the range of about 0.01% to about 10% of the total injectable composition of the peptide drug.

17. The injectable composition according to claim 1, wherein the pH is between about 3.0 and about 13.0.

18. The injectable composition according to claim 1, wherein the antioxidant is added in an amount preferably from 0.001 to 1% w/v.

19. A process for the preparation of a stable, non-aqueous and ready-to-use injectable composition of a pharmaceutically active agent or a pharmaceutically acceptable salt or a co-crystal thereof; comprising the steps of:
a) dissolving a pH adjusting agent in a non-aqueous solvent system consisting of a primary non-aqueous solvent to obtain a first solution;
b) optionally adding polyol and antioxidant to the secondary non-aqueous solvent under constant stirring until the polyol dissolves to obtain a second solution;
c) adding the first solution of step (a) to the second solution of step (b) under constant stirring to obtain a third solution;
d) dispersing the pharmaceutically active agent in the third solution of step (c) to obtain a dispersion;
e) optionally filtering the dispersion of step (d) to obtain a clear solution; and
f) filling the clear solution of step (e) into a container to obtain a preparation in a ready-to-use form.

20. A process for the preparation of a stable, non-aqueous and ready-to-use injectable composition of a pharmaceutically active agent or a pharmaceutically acceptable salt or a co-crystal thereof comprising the steps of:
a) dissolving polyol, optionally antioxidant and pH adjusting agent in secondary non-aqueous solvent to obtain a first solution;
b) adding primary non-aqueous solvent to the first solution of step (a) to obtain a second solution;
c) adding pharmaceutically active agent to the second solution of step (b) and allowing to disperse to produce a dispersion;
d) optionally filtering the dispersion of step (c) one or more times to obtain a clear solution; and
e) filling the clear solution of step (d) into a container to obtain a composition in a ready-to-use form.

21. A method for the treatment of one or more diseases, disorders or conditions, comprising administering to a subject in need thereof; the injectable composition as claimed in claim 1 in an amount effective to treat the conditions, diseases or disorders.
